(19) United States
(12) Patent Application Publication
(10) Pub. No.: US 2020/0218826 A1
(43) Pub. Date: Jul. 9, 2020

(54) DATA SEARCHING SYSTEM, DATA SEARCHING METHOD AND COMPUTER READABLE MEDIUM

(71) Applicant: MITSUBISHI ELECTRIC CORPORATION, Tokyo (JP)

(72) Inventors: Sachio NOHARA, Amagasaki-shi (JP); Shigeki TANISHIMA, Amagasaki-shi (JP); Takato HIRANO, Tokyo (JP)

(73) Assignee: MITSUBISHI ELECTRIC CORPORATION, Tokyo (JP)

(21) Appl. No.: 16/647,857

(22) PCT Filed: Sep. 4, 2018
(86) PCT No.: PCT/JP2018/032706

§ 371 Date: Mar. 16, 2020

(30) Foreign Application Priority Data
Sep. 21, 2017 (JP) 2017-180966

(51) Int. Cl. G06F 21/62; G16H 10/00; H04L 9/08; G06F 16/14

(52) U.S. Cl. CPC G06F 21/6245; G06F 21/6227; G06F 16/156; H04L 9/0825; G06F 16/148; G16H 10/00

(57) ABSTRACT

In a personal searching-purpose ID (511), a personal encryption ID (512) and encrypted personal information (513) stored in a personal information storage unit (51), a disclosure range of personal information is embedded. Also, in a medical searching-purpose ID and a medical encryption ID stored in a medical data storage unit, a disclosure range of medical data is embedded. A search query generation unit generates a search query Q by encrypting a search anonymous ID subject to searching acquired from a user with attribution information of the user embedded. Then, a searching unit executes confidential searching on the personal searching-purpose ID (511) and a pathological searching-purpose ID (521), using the search query Q. The searching unit outputs a searching result acquired based on the attribution information of the user, the disclosure range of personal information and the disclosure range of medical data.

### TECHNICAL FIELD

The present invention relates to a medical data searching system, a medical data searching method and a medical data searching program. Especially, the present invention relates to the medical data searching system, the medical data searching method and the medical data searching program for confidentially searching medical data such as pathological diagnosis information or genetic diagnosis information.

### BACKGROUND ART

In recent years, it has been possible to perform a genetic analysis at a low cost. On the other hand, it is essential to compare genetic information with genetic information of various kinds of people or to analyze the genetic information of various kinds of people for an appropriate diagnosis and increasing knowledge of the genetic analysis.

When handling medical data such as the genetic information, it is necessary to consider privacy. There is a method in which data is analyzed while keeping the data encrypted, but it takes a long time for an analysis because the medical data has a large amount of data. Therefore, it is difficult to apply the method in which data is analyzed while keeping the data encrypted, to the medical data at the present situation. Accordingly, there is an increasing demand for an anonymous ID (IDentifier) management technology extracting the medical data as necessary while at first glance it is not clear whose medical data it is, though the medical data is a plaintext.

The anonymous ID management technology is a technology which manages data by assigning a temporary ID not a personal name. Anyone can calculate a hash value when managing data by the temporary ID which uses the hash value generated deterministically. Thus, there is a risk that the personal name is guessed by calculations based on experiments with various hash value inputs. Also, in a technology which uses a ciphertext of a general common key encryption, it is necessary to provide all the data registrants with the same key, which increases a risk of key leakage. Also, in a technology which uses a ciphertext of a general public key encryption, management of a secret key and a ciphertext dependent on the number of users is required, and the number of managed objects becomes enoiiuous.

In Patent Literature 1, a method is disclosed in which the medical data is managed while keeping the data encrypted, using an encryption technology called a confidential searching technology. In the technology of Patent Literature 1, the temporary ID is encrypted stochastically, but it is possible to be combined by using a search query. In the technology of Patent Literature 1, an authorized user such as an attending physician can concatenate personal information to the medical data.

### CITATION LIST

### Patent Literature

Patent Literature 1: JP2015-022395A

### SUMMARY OF INVENTION

### Technical Problem

In Patent Literature 1, control such as data disclosure and data concatenation according to a user is difficult. Also in Patent Literature 1, as the number of users increases, the number of public key and secret key pairs and ciphertexts also increases, thereby increasing burdens of key management and information management.

The present invention aims at providing a medical data searching system which enables the data disclosure and the data concatenation according to the user while reducing burdens of the key management and the information management.

### Solution to Problem

A medical data searching system according to the present invention includes,

a management device including:

a personal information storage unit to store a personal searching-purpose ID, a personal encryption ID and encrypted personal information, the personal searching-purpose ID being a personal searching-purpose ID used for confidential searching and obtained by encrypting an anonymous ID (IDentifier) for identifying personal information with a disclosure range of the personal information embedded, the personal encryption ID and the encrypted personal information being obtained by encrypting the anonymous ID and the personal information with the disclosure range of the personal information embedded; and

a medical data storage unit to store a medical searching-purpose ID, a medical encryption ID and medical data, the medical searching-purpose ID being a medical searching-purpose ID used for confidential searching and obtained by encrypting the anonymous ID with a disclosure range of the medical data corresponding to the personal information embedded, the medical encryption ID being obtained by encrypting the anonymous ID with the disclosure range of the medical data embedded; and

a searching device including:

a search query generation unit to acquire the anonymous ID subject to searching from a user as a searching anonymous ID, and generate a search query obtained by encrypting the searching anonymous ID with attribution information of the user embedded; and

a searching unit to execute confidential searching on the personal searching-purpose ID and the medical searching-purpose ID, using the search query, and output a searching result acquired based on the attribution information of the user, the disclosure range of the personal information and the disclosure range of the medical data.

The medical data searching system includes,

a key management device including:

a confidential searching-purpose key storage unit to store a confidential searching-purpose public key and a confidential searching-purpose secret key with the attribution information of the user embedded;

an encryption-purpose key storage unit to store an encryption-purpose public key and an encryption-purpose secret key with the attribution information of the user embedded;

an information storage unit to store authority setting information including the disclosure range of the personal information and the disclosure range of the medical data; and

a public key information transmission unit to transmit public key information including the confidential searching-purpose public key, the encryption-purpose public key and the authority setting information.

The medical data searching system includes,

a personal information registration device including:

a personal searching-purpose encryption unit to encrypt the anonymous ID as the personal searching-purpose ID with the disclosure range of the personal information embedded, using the confidential searching-purpose public key and the authority setting information included in the public key information; and

a personal decryption-purpose encryption unit to encrypt the personal information and the anonymous ID as the encrypted personal information and the personal encryption ID with the disclosure range of the personal information embedded, using the encryption-purpose public key and the authority setting information included in the public key information.

The medical data searching system includes,

a medical information registration device including:

a medical searching-purpose encryption unit to encrypt the anonymous ID as the medical searching-purpose ID with the disclosure range of the medical data embedded, using the confidential searching-purpose public key and the authority setting information included in the public key information; and a medical decryption-purpose encryption unit to encrypt the anonymous ID as the medical encryption ID with the disclosure range of the medical data embedded, using the encryption-purpose public key and the authority setting information included in the public key information.

The search query generation unit,

generates the search query with the attribution information of the user embedded, using the confidential searching-purpose secret key.

The searching unit,

outputs the personal encryption ID and the encrypted personal information corresponding to the personal searching-purpose ID in which the attribution information of the user embedded in the search query satisfies the disclosure range of the personal information, as the searching result.

The searching unit,

outputs the medical encryption ID and the medical data corresponding to the medical searching-purpose ID in which the attribution information of the user embedded in the search query satisfies the disclosure range of the medical data, as the searching result.

The medical data storage unit,

stores the medical searching-purpose ID, the medical encryption ID and the medical data, the medical searching-purpose ID being the medical searching-purpose ID obtained by encrypting the anonymous ID, and indicating if the medical data may be used for a research-purpose or not, the medical encryption ID being the medical encryption ID obtained by encrypting the anonymous ID, and indicating if the medical data may be used for a research-purpose or not.

The medical data searching system includes,

an information generation unit to decrypt the personal encryption ID and the medical encryption ID output as the searching result, and combine as result information, the personal encryption ID and the medical encryption ID output as the searching result, when decrypting results of the personal encryption ID and the medical encryption ID are equal.

The information generation unit,

decrypts the result information to reference information, using the encryption-purpose secret key.

A medical data searching method includes:

storing, by the personal information storage unit of the management device, the personal searching-purpose ID, the personal encryption ID and the encrypted personal information, the personal searching-purpose ID being a personal searching-purpose ID used for confidential searching and obtained by encrypting an anonymous ID (IDentifier) for identifying the personal information with the disclosure range of the personal information embedded, the personal encryption ID and the encrypted personal information being obtained by encrypting the anonymous ID and the personal information with the disclosure range of the personal information embedded;

storing, by the medical data storage unit of the management device, the medical searching-purpose ID, the medical encryption ID and the medical data, the medical searching-purpose ID being a medical searching-purpose ID used for confidential searching and obtained by encrypting the anonymous ID with the disclosure range of the medical data corresponding to the personal information embedded, the medical encryption ID being obtained by encrypting the anonymous ID with the disclosure range of the medical data embedded;

acquiring, by the search query generation unit of the searching device, the anonymous ID subject to searching from a user as a searching anonymous ID, and generating the search query obtained by encrypting the searching anonymous ID with attribution information of the user embedded; and

executing, by the searching unit of the searching device, confidential searching on the personal searching-purpose ID and the medical searching-purpose ID, using the search query, and outputting the searching result acquired based on the attribution information of the user, the disclosure range of the personal information and the disclosure range of the medical data.

A medical data searching program for a searching device,

the searching device, that is a computer, searching in the personal information storage unit and the medical data storage unit, the personal information storage unit storing the personal searching-purpose ID, the personal encryption ID and the encrypted personal information, the personal searching-purpose ID being a personal searching-purpose ID used for confidential searching and obtained by encrypting an anonymous ID (IDentifier) for identifying the personal information with the disclosure range of the personal information embedded, the personal encryption ID and the encrypted personal information being obtained by encrypting the anonymous ID and the personal information with the disclosure range of the personal information embedded, the medical data storage unit storing the medical searching-purpose ID, the medical encryption ID and the medical data, the medical searching-purpose ID being a medical searching-purpose ID used for confidential searching and obtained by encrypting the anonymous ID with the disclosure range of the medical data corresponding to the personal information embedded, the medical encryption ID being obtained by encrypting the anonymous ID with the disclosure range of the medical data embedded,

the medical data searching program causing the searching device to execute:

a search query generation process of acquiring the anonymous ID subject to searching from a user as the searching anonymous ID, and generating the search query obtained by encrypting the searching anonymous ID with attribution information of the user embedded; and

a confidential searching process of executing confidential searching on the personal searching-purpose ID and the medical searching-purpose ID, using the search query, and outputting the searching result acquired based on the attribution information of the user, the disclosure range of the personal information and the disclosure range of the medical data.

### Advantageous Effects of Invention

In a medical data searching system according to the present invention, a personal information storage unit stores a personal searching-purpose ID used for confidential searching, a personal encryption ID for decryption, and encrypted personal information. In the personal searching-purpose ID, the personal encryption ID and the encrypted personal information, a disclosure range of personal information is embedded. Also, a medical data storage unit stores a medical searching-purpose ID used for confidential searching and a medical encryption ID for decryption. In the medical searching-purpose ID and the medical encryption ID, a disclosure range of medical data corresponding to the personal information is embedded. A search query generation unit generates a search query by encrypting a searching anonymous ID subject to searching acquired from a user with attribution information of the user embedded. Then, a searching unit executes confidential searching on the personal searching-purpose ID and the medical searching-purpose ID, using the search query. The searching unit outputs a searching result acquired based on the attribution information of the user, the disclosure range of the personal information and the disclosure range of the medical data. According to the medical data searching system of the present invention, it is possible to execute confidential searching with access control based on the attribution information of the user, the disclosure range of the personal information and the disclosure range of the medical data. Consequently, reference control on the personal information and the medical data according to the attribution information of the user can be realized.

### BRIEF DESCRIPTION OF DRAWINGS

FIG. 1 is a configuration diagram of a medical data searching system 100 according to Embodiment 1;

FIG. 2 is a configuration diagram of a key management device 200 according to Embodiment 1;

FIG. 3 is a configuration diagram of a management device 500 according to Embodiment 1;

FIG. 4 is a configuration diagram of a personal information registration device 310 according to Embodiment 1;

FIG. 5 is a configuration diagram of a medical data registration device 320 according to Embodiment 1;

FIG. 6 is a configuration diagram of a searching device 400 according to Embodiment 1;

FIG. 7 is a diagram illustrating an example of a hardware configuration of each device of the key management device 200, the personal information registration device 310, the medical data registration device 320, the searching device 400 and the management device 500 according to Embodiment 1;

FIG. 8 is a flowchart of a personal information registration process S110 according to Embodiment 1;

FIG. 9 is a flowchart of a medical data registration process S120 according to Embodiment 1;

FIG. 10 is a schematic diagram illustrating the personal information registration process S110 and the medical data registration process S120 according to Embodiment 1;

FIG. 11 is a flowchart of a searching process S130 according to Embodiment 1;

FIG. 12 is a schematic diagram illustrating a case in which an attending physician searches the management device 500 as a user;

FIG. 13 is a schematic diagram illustrating a case in which a genetic counselor searches the management device 500 as a user;

FIG. 14 is a schematic diagram illustrating a case in which a researcher searches the management device 500 as a user.

### DESCRIPTION OF EMBODIMENTS

An embodiment of the present invention will be described below, using diagrams. In each diagram, the same reference signs are provided to the same elements or corresponding elements. In descriptions of the embodiment, descriptions of the same elements or corresponding elements are omitted or simplified as appropriate.

### Embodiment 1

***Description of Configuration***

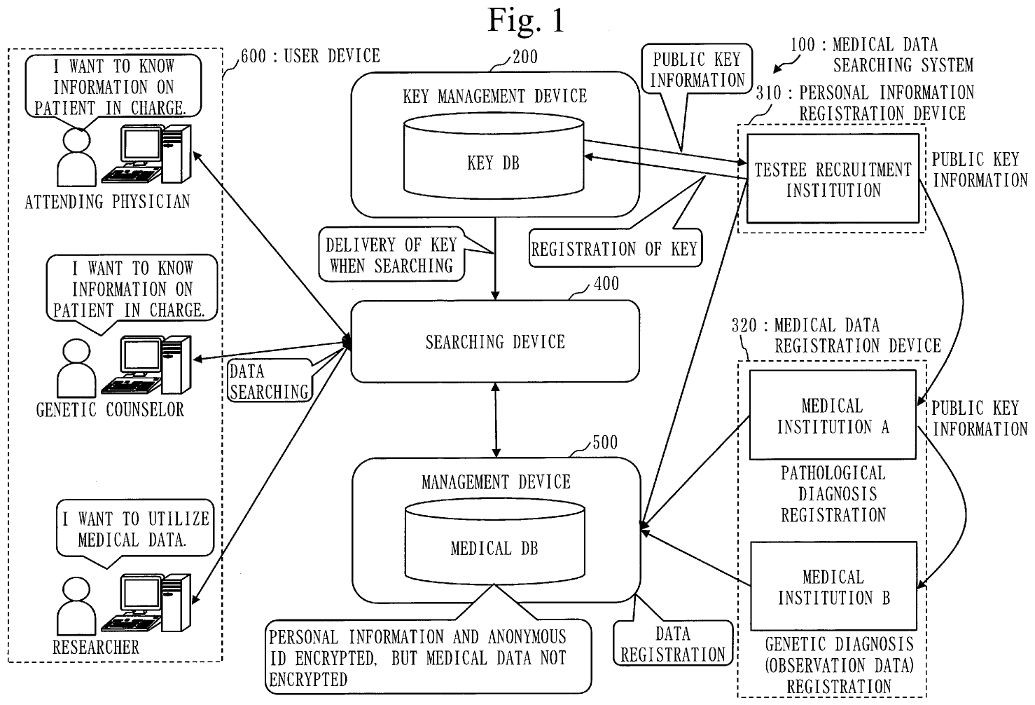

An outline of a configuration of a medical data searching system 100 according to the present embodiment will be described, using FIG. 1. In the medical data searching system 100, medical data is managed by an anonymous ID management technology. The anonymous ID is an anonymous ID for identifying personal information. The personal information is information such as a full name, age, address of a person. The medical data is information such as pathological diagnosis information and genetic diagnosis information, the pathological diagnosis information being information on a pathological diagnosis received by a person at a medical institution, the genetic diagnosis information being observation data provided by the medical institution.

The medical data searching system 100 includes a key management device 200, a personal information registration device 310, a medical data registration device 320, a searching device 400, a management device 500 and a user device 600. The key management device 200, the personal information registration device 310, the medical data registration device 320, the searching device 400, the management device 500 and the user device 600 are connected via a network. Specifically, the network is the Internet or a LAN (Local Area Network), and also networks of other kinds may be used. Additionally, each device of the medical data searching system 100 may be connected without using the network. Also, a plurality of devices out of devices in the medical data searching system 100 may be installed in one computer.

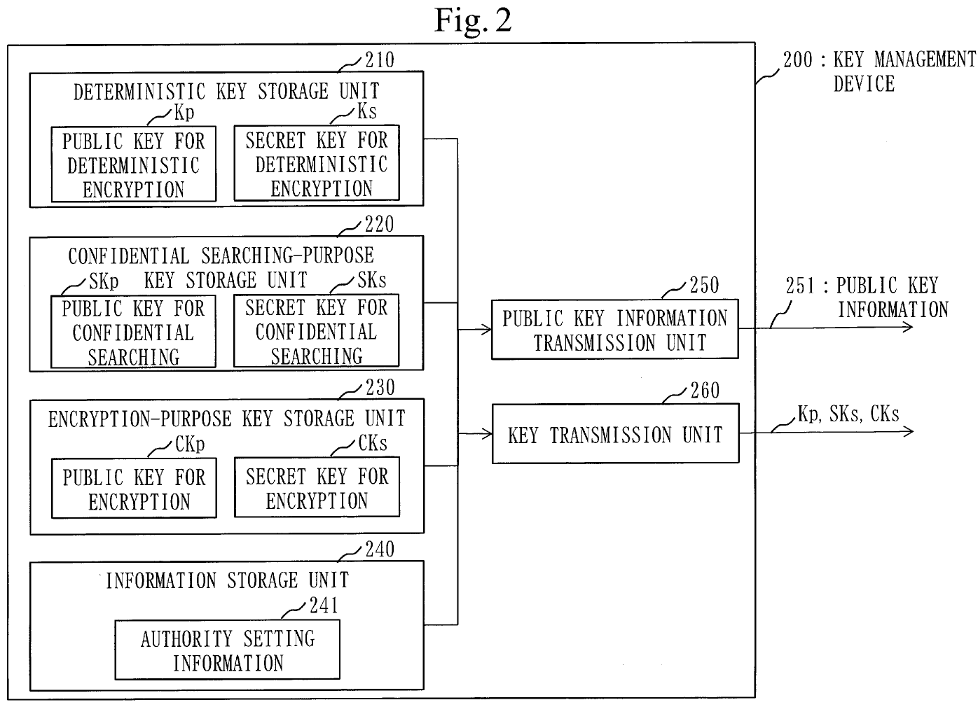

A configuration of the key management device 200 according to the present embodiment will be described, using FIG. 2.

The key management device 200 is a computer. The key management device 200 includes a deterministic key storage unit 210, a confidential searching-purpose key storage unit 220, an encryption-purpose key storage unit 230, an information storage unit 240, a public key information transmission unit 250 and a key transmission unit 260.

The deterministic key storage unit 210 stores a public key Kp and a secret key Ks for deterministic encryption. The public key Kp and the secret key Ks for deterministic encryption are used for encryption and decryption of the anonymous ID.

The confidential searching-purpose key storage unit 220 stores a public key SKp for confidential searching, and a secret key SKs for confidential searching in which attribution information of a user is embedded. The attribution information of the user is, for example, information indicating an occupation of the user. Specifically, the attribution information of the user is information indicating an occupation dealing with the medical data, such as “attending physician”, “genetic counselor” and “researcher”. The public key SKp stored in the confidential searching-purpose key storage unit 220 is used when encrypting a word to be registered together with the attribution information of the user who is allowed to search for the word to be registered on an occasion of registering data in the management device 500. Also, the secret key SKs stored in the confidential searching-purpose key storage unit 220 is used for encrypting data to be searched when searching encrypted data registered in the management device 500.

If the attribution information of the user to be included in the registered data when encrypting the registered data and the attribution information of the user included in the secret key SKs coincide with each other, and if the registered data and the searched data are the same, it is possible to determine that these pieces of data coincide with each other while keeping them encrypted. On the other hand, if the attribution information of the user to be included in the registered data when encrypting the registered data and the attribution information of the user included in the secret key SKs are different, it is determined that these pieces of data do not coincide with each other even when the registered data and the searching data are the same.

The encryption-purpose key storage unit 230 stores a public key CKp for encryption, and a secret key CKs for encryption in which the attribution information of the user is embedded. The public key CKp stored in the encryption-purpose key storage unit 230 is used when encrypting a word to be registered together with the attribution information of the user who is allowed to search for the word to be registered on an occasion of registering data in the management device 500. The secret key CKs stored in the encryption-purpose key storage unit 230 is used for decrypting the encryption data registered in the management device 500.

When the attribution information of the user included in the registered encryption data and the attribution information of the user included in the secret key CKs coincide with each other, the encryption data can be decrypted.

The information storage unit 240 stores authority setting information 241 which includes a disclosure range of the personal information and a disclosure range of medical data.

The public key information transmission unit 250 transmits public key information 251 which includes the public key SKp for confidential searching, the public key CKp for encryption and the authority setting information 241. The public key information 251 also includes the public key Kp for deterministic encryption.

The key transmission unit 260 transmits to the searching device 400, the public key Kp for deterministic encryption and secret keys SKs, CKs corresponding to the attribution information of the user.

The key management device 200 may, for example, acquire a parameter from the user, and generate the public key Kp and the secret key Ks for deterministic encryption, the public key SKp and the secret key SKs used for searching, and the public key CKp and the secret key Cks used for encryption. Alternatively, the key management device 200 may acquire a key generated outside of the key management device 200, and store it inside. Specifically, the key management device 200 may acquire a key generated by the personal information registration device 310, and store it inside.

The deterministic key storage unit 210, the confidential searching-purpose key storage unit 220 and the encryption-purpose key storage unit 230 are examples of a key DB (Data Base).

Also in the authority setting information 241, the following information is included, for example.

As illustrated in FIG. 1, the user device 600 is specifically a device used by an attending physician, a genetic counselor and a researcher. Each of the attending physician, the genetic counselor and the researcher has authority as below.

The attending physician can concatenate personal information, pathological diagnosis information and genetic diagnosis information of a patient, and refer to them.

The genetic counselor can concatenate the personal information and the genetic diagnosis information of the patient, and refer to them, but cannot refer to the pathological diagnosis information of the patient.

The researcher is a user who secondarily uses the medical data. The researcher can concatenate the pathological diagnosis information and the genetic diagnosis information of the patient, and refer to them with the patient's consent. However, the researcher cannot concatenate the pathological diagnosis information and the genetic diagnosis information without the patient's consent.

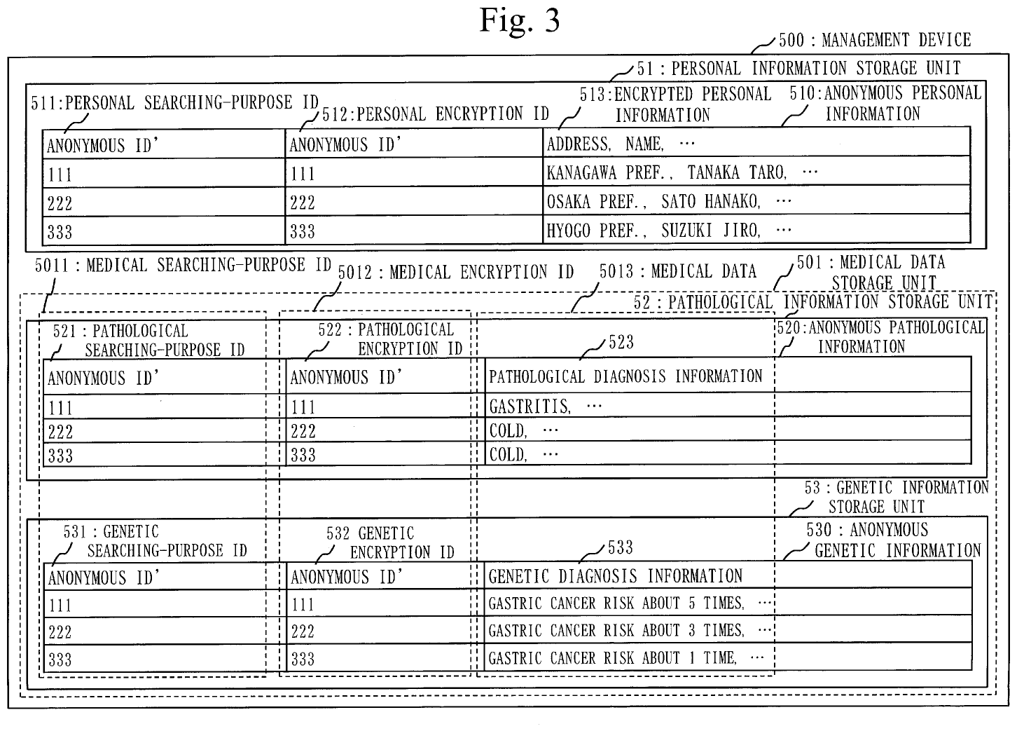

A configuration of the management device 500 according to the present embodiment is described, using FIG. 3.

The management device 500 is specifically a computer including a large-capacity storage device. The management device 500 includes a personal information storage unit 51 and a medical data storage unit 501. The medical data storage unit 501 includes a pathological information storage unit 52 and a genetic information storage unit 53.

In the personal information storage unit 51, anonymous personal information 510 is stored. In the pathological information storage unit 52, anonymous pathological information 520 is stored. In the genetic information storage unit 53, anonymous genetic information 530 is stored.

In the anonymous personal information 510, a personal searching-purpose ID 511, a personal encryption ID 512 and encrypted personal information 513 are correlated. The personal searching-purpose ID 511 is used for confidential searching. The personal searching-purpose ID 511 is information obtained by encrypting the anonymous ID for identifying the personal information with the disclosure range of the the personal information embedded. Also, the personal encryption ID 512 and the encrypted personal information 513 are information obtained by encrypting the anonymous ID and the personal information with the disclosure range of the personal information embedded.

When the personal searching-purpose ID 511 is extracted by confidential searching, the personal encryption ID 512 is decrypted and used when concatenating the personal information, the pathological diagnosis information and the genetic diagnosis information.

In the anonymous pathological information 520, a pathological searching-purpose ID 521, a pathological encryption ID 522 and pathological diagnosis information 523 are correlated. The pathological searching-purpose ID 521 is used for confidential searching. The pathological searching-purpose ID 521 is information obtained by encrypting the anonymous ID with a disclosure range of the pathological diagnosis information 523 embedded, the pathological diagnosis information 523 corresponding to the personal information. The pathological encryption ID 522 is information obtained by encrypting the anonymous ID with the disclosure range of the pathological diagnosis information 523 embedded. The pathological diagnosis information 523 that is the medical data, is stored without being encrypted.

When the pathological searching-purpose ID 521 is extracted by confidential searching, the pathological encryption ID 522 is decrypted and used when concatenating the personal information, the pathological diagnosis information and the genetic diagnosis information.

In the anonymous genetic information 530, a genetic searching-purpose ID 531, a genetic encryption ID 532 and genetic diagnosis information 533 are correlated. The genetic searching-purpose ID 531 is used for confidential searching. The genetic searching-purpose ID 531 is information obtained by encrypting the anonymous ID with a disclosure range of the genetic diagnosis information 533 embedded, the genetic diagnosis information 533 corresponding to the personal information. The genetic encryption ID 532 is information obtained by encrypting the anonymous ID with the disclosure range of the genetic diagnosis information 533 embedded. The genetic diagnosis information 533 that is the medical data, is stored without being encrypted.

When the genetic searching-purpose ID 531 is extracted by confidential searching, the genetic encryption ID 532 is decrypted and used when concatenating the personal information, the pathological diagnosis information and the genetic diagnosis information.

The pathological searching-purpose ID 521 and the genetic searching-purpose ID 531 are examples of a medical searching-purpose ID 5011. The pathological encryption ID 522 and the genetic encryption ID 532 are examples of a medical encryption ID 5012. Also, the personal information storage unit 51 and the medical data storage unit 501 are examples of a medical DB.

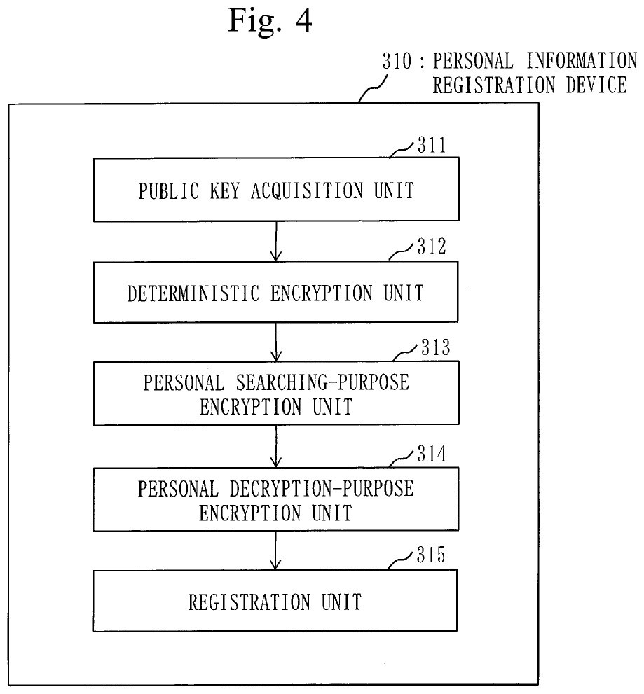

A configuration of the personal information registration device 310 will be described, using FIG. 4.

The personal information registration device 310 registers the personal information in the management device 500. The personal information registration device 310 is specifically a device of a testee recruitment institution. Note that the personal information registration device 310 that is the device of the testee recruitment institution, may register a key in the key management device 200.

The personal information registration device 310 includes a public key acquisition unit 311, a deterministic encryption unit 312, a personal searching-purpose encryption unit 313, a personal decryption-purpose encryption unit 314 and a registration unit 315.

The public key acquisition unit 311 acquires the public key information 251 from the key management device 200. In the public key information 251, the public key Kp, the public key SKp for confidential searching, the public key CKp for encryption and the authority setting information 241 are included.

The deterministic encryption unit 312 encrypts the anonymous ID into an anonymous ID′, using the public key Kp.

The personal searching-purpose encryption unit 313 encrypts the anonymous ID′ as the personal searching-purpose ID 511 with the disclosure range of the personal information embedded, using the public key SKp for confidential searching and the authority setting information 241 included in the public key information 251.

The personal decryption-purpose encryption unit 314 encrypts the personal information and the anonymous ID′ as the encrypted personal information 513 and the personal encryption ID 512 with the disclosure range of the personal information embedded, using the public key CKp for encryption and the authority setting information 241 included in the public key information 251.

The registration unit 315 registers the personal searching-purpose ID 511, the personal encryption ID 512 and the encrypted personal information 513 in the management device 500.

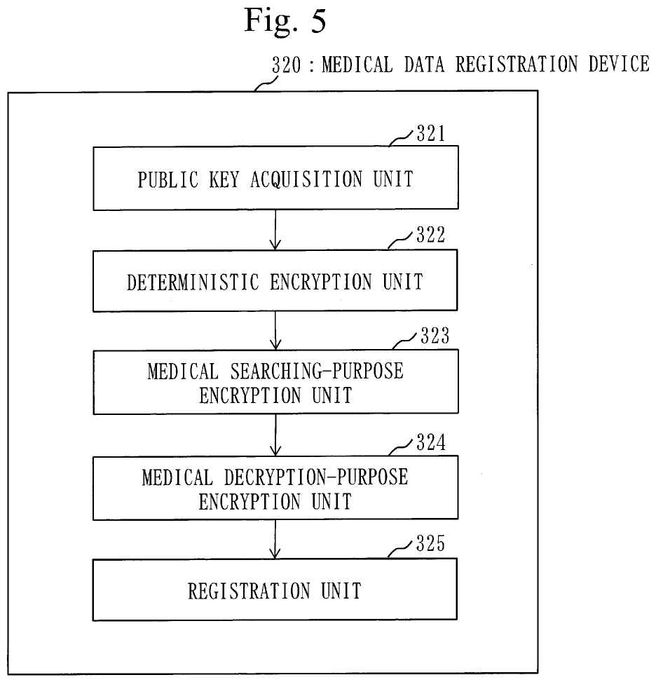

A configuration of the medical data registration device 320 according to the present embodiment will be described, using FIG. 5.

The medical data registration device 320 registers the medical data in the management device 500. The medical data registration device 320 is specifically each device of a plurality of medical institutions. The medical data registration device 320 is each device of the plurality of medical institutions such as a device of a medical institution A registering the pathological diagnosis information in the management device 500, a device of a medical institution B registering the genetic diagnosis information in the management device 500, for example.

The medical data registration device 320 includes a public key acquisition unit 321, a deterministic encryption unit 322, a medical searching-purpose encryption unit 323, a medical decryption-purpose encryption unit 324 and a registration unit 325.

The public key acquisition unit 321 acquires the public key information 251 from the key management device 200 or the personal information registration device 310. The public key information 251 includes the public key Kp, the public key SKp for confidential searching, the public key CKp for encryption and the authority setting information 241.

The deterministic encryption unit 322 encrypts the anonymous ID into the anonymous ID′ using the public key Kp.

As mentioned above, the medical institution A deals with the pathological diagnosis information 523. Therefore, a function of the medical data registration device 320 of the medical institution A is as follows.

The medical searching-purpose encryption unit 323 encrypts the anonymous ID′ as the pathological searching-purpose ID 521 with a disclosure range of the pathological diagnosis information 523 embedded, using the public key SKp for confidential searching and the authority setting information 241 included in the public key information 251.

The medical decryption-purpose encryption unit 324 encrypts the anonymous ID′ as the pathological encryption ID 522 with the disclosure range of the pathological diagnosis information 523 embedded, using the public key CKp for encryption and the authority setting information 241 included in the public key information 251. The registration unit 325 registers the pathological searching-purpose ID 521, the pathological encryption ID 522 and the pathological diagnosis information 523 in the management device 500.

Also, as described above, the medical institution B deals with the genetic diagnosis information 533. Therefore, a function of the medical data registration device 320 of the medical institution B is as follows.

The medical searching-purpose encryption unit 323 encrypts the anonymous ID′ as the genetic searching-purpose ID 531 with a disclosure range of the genetic diagnosis information 533 embedded, using the public key SKp for confidential searching and the authority setting information 241 included in the public key information 251.

The medical decryption-purpose encryption unit 324 encrypts the anonymous ID′ as the genetic encryption ID 532 with the disclosure range of the genetic diagnosis information 533 embedded, using the public key CKp for encryption and the authority setting information 241 included in the public key information 251. The registration unit 325 registers the genetic searching-purpose ID 531, the genetic encryption ID 532 and the genetic diagnosis information 533 in the management device 500.

The pathological searching-purpose ID 521 and the genetic searching-purpose ID 531 are examples of the medical searching-purpose ID 5011. The pathological encryption ID 522 and the genetic encryption ID 532 are examples of the medical encryption ID 5012.

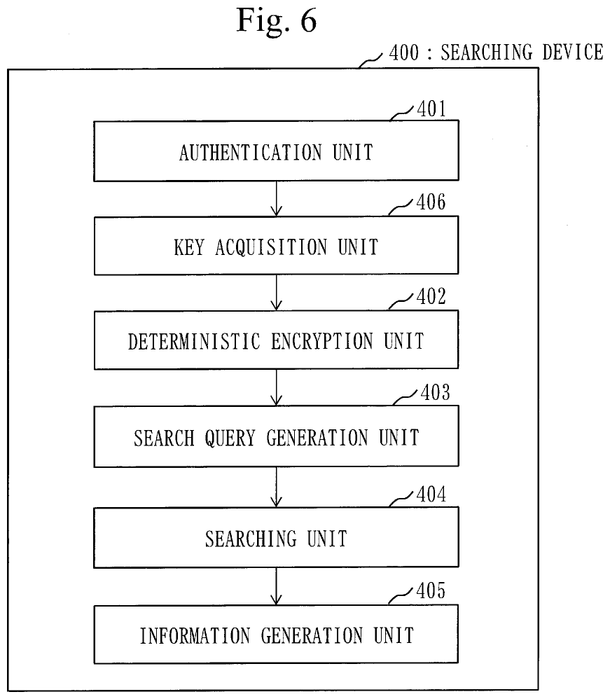

A configuration of the searching device 400 according to the present embodiment will be described, using FIG. 6.

The searching device 400 includes an authentication unit 401, a key acquisition unit 406, a deterministic encryption unit 402, a search query generation unit 403, a searching unit 404 and an information generation unit 405.

The authentication unit 401 acquires from the user device 600, user information for authenticating the user, and authenticates the user.

The key acquisition unit 406 requires the key management device 200 to provide the public key Kp for deterministic encryption and secret keys SKs and CKs corresponding to the attribution information of the user. Then, the key acquisition unit 406 acquires the public key Kp and secret keys SKs and CKs transmitted from the key management device 200.

The deterministic encryption unit 312 acquires the anonymous ID subject to searching, as the search anonymous ID, from the user, and encrypts the searching anonymous ID, using the public key Kp.

The search query generation unit 403 acquires the anonymous ID subject to searching, as the search anonymous ID, from the user, and generates a search query Q obtained by encrypting the search anonymous ID with the attribution information of the user embedded. The search query generation unit 403 generates the search query Q in which the attribution information of the user is embedded, using the secret key SKs for confidential searching.

The searching unit 404 executes confidential searching on the personal searching-purpose ID 511 and the medical searching-purpose ID 5011, using the search query Q. The searching unit 404 outputs the searching result acquired based on the attribution information of the user, the disclosure range of the personal information and the disclosure range of the medical data.

The information generation unit 405 decrypts the personal encryption ID 512 and the medical encryption ID 5012 output as the searching result, using the secret key CKs. The information generation unit 405 combines the searching result output from the searching unit 404 as result information, when decryption results of the personal encryption ID 512 and the medical encryption ID 5012 are equal. The information generation unit 405 decrypts the result information to plaintext reference information, using the secret key CKs.

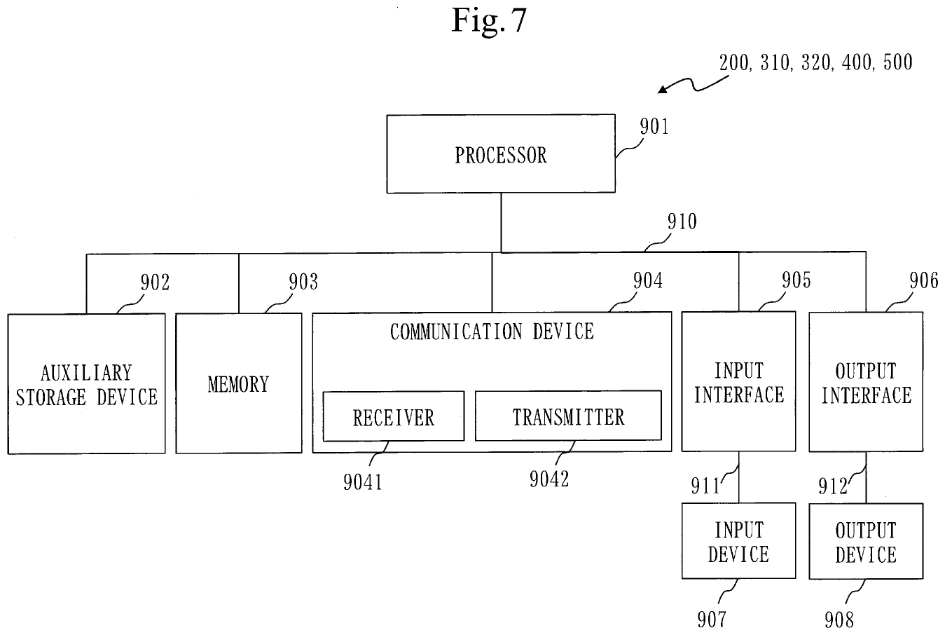

An example of a hardware configuration of each device of the key management device 200, the personal information registration device 310, the medical data registration device 320, the searching device 400 and the management device 500 is described, using FIG. 7. Hereinbelow, each device of the key management device 200, the personal information registration device 310, the medical data registration device 320, the searching device 400 and the management device 500 may be referred to as each device in the medical data searching system 100 below. Also, each unit of each device of the medical data searching system 100 illustrated in FIG. 2 to FIG. 6 may be referred to as “unit” of each device in the medical data searching system 100. Note that the “unit” of each device does not include a “storage unit”.

Each device of the key management device 200, the personal information registration device 310, the medical data registration device 320, the searching device 400 and the management device 500 is a computer.

Each device of the key management device 200, the personal information registration device 310, the medical data registration device 320, the searching device 400 and the management device 500 includes hardware such as a processor 901, an auxiliary storage device 902, a memory 903, a communication device 904, an input interface 905 and an output interface 906.

The processor 901 is connected with other hardware components via a signal line 910, and controls these other hardware components.

The input interface 905 is connected to an input device 907.

The output interface 906 is connected to an output device 908.

The processor 901 is an IC (Integrated Circuit) which performs a calculation process. Specific examples of the processor 901 are a CPU (Central Processing Unit), a DSP (Digital Signal Processor) and a GPU (Graphics Processing Unit).

Specific examples of the auxiliary storage device 902 are a ROM (Read Only Memory), a flash memory, an HDD (Hard Disk Drive).

A specific example of the memory 903 is a RAM (Random Access Memory).

The communication device 904 includes a receiver 9041 which receives data and a transmitter 9042 which transmits data. Specific examples of the communication device 904 are a communication chip or an NIC (Network Interface Card).

The input interface 905 is a port to which a cable 911 of the input device 907 is connected. A specific example of the input interface 905 is a USB (Universal Serial Bus) terminal.

The output interface 906 is a port to which a cable 912 of the output device 908 is connected. Specific examples of the output interface 906 are the USB terminal and an HDMI (registered trademark) (High Definition Multimedia Interface) terminal.

Specific examples of the input device 907 are a mouse, a keyboard and a touch panel.

A specific example of the output device 908 is a display, and for example an LCD (Liquid Crystal Display).

In the auxiliary storage device 902 of each device, programs for realizing functions of “units” of each device are stored. Note that “storage units” in each device are stored in the auxiliary storage device 902 or the memory 903.

The programs for realizing functions of “units” may be one program, or may be composed of a plurality of programs.

This program is loaded to the memory 903, read by the processor 901, and executed by the processor 901.

In addition, an OS (Operating System) is stored in the auxiliary storage device 902. At least a part of the OS is loaded to the memory 903, and the processor 901 executes programs for realizing functions of “units” while executing the OS.

In FIG. 7, one processor 901 is illustrated, but each device may include a plurality of processors 901. Additionally, the plurality of processors 901 may execute the programs for realizing functions of “units” in cooperation.

Also, at least any of information, data, a signal value, or a variable value indicating processing results of “units” is stored in the memory 903, the auxiliary storage device 902, or a register or a cache memory of the processor 901.

Also, the programs for realizing functions of “units” are stored in a storage medium such as a magnetic disk, a flexible disk, an optical disk, a compact disk, a Blu-ray (registered trademark) or a DVD.

“Units” may be provided as “processing circuitry”.

Also, “units” may be read as “circuits”, “steps”, “procedures” or “processes”.

“Circuits” and “processing circuitry” are the concept including not only the processor 901, but also other kinds of processing circuitry such as a logic IC, a GA (Gate Array), an ASIC (Application Specific Integrated Circuit) or an FPGA (Field-Programmable Gate Array).

***Description of Operation***

Next, a medical data searching method 610 in the medical data searching system 100, and a medical data searching process S100 by a medical data searching program 620 according to the present embodiment will be described.

The medical data searching process S100 includes a personal information registration process S110, a medical data registration process S120 and a searching process S130.

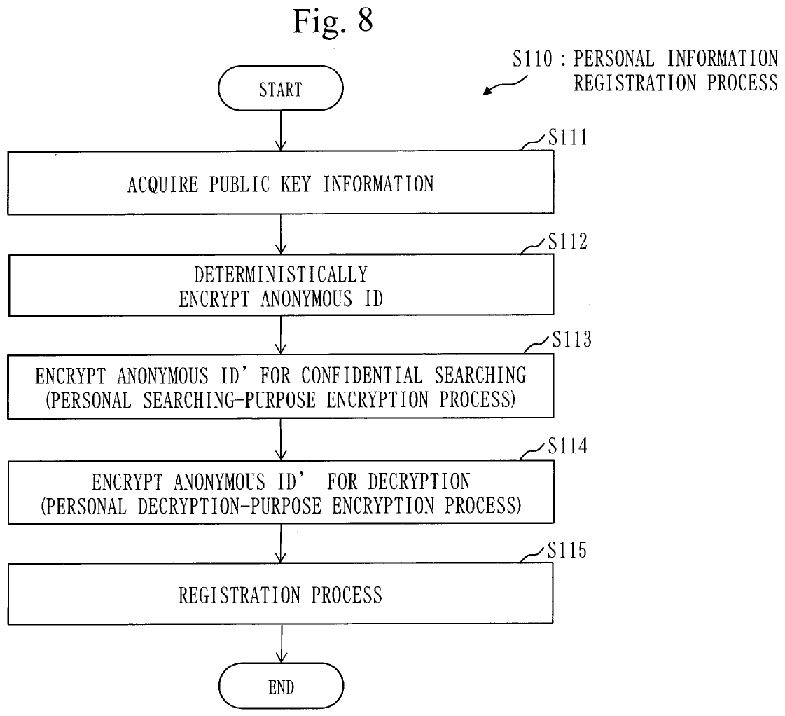

FIG. 8 is a flowchart of the personal information registration process S110 according to the present embodiment.

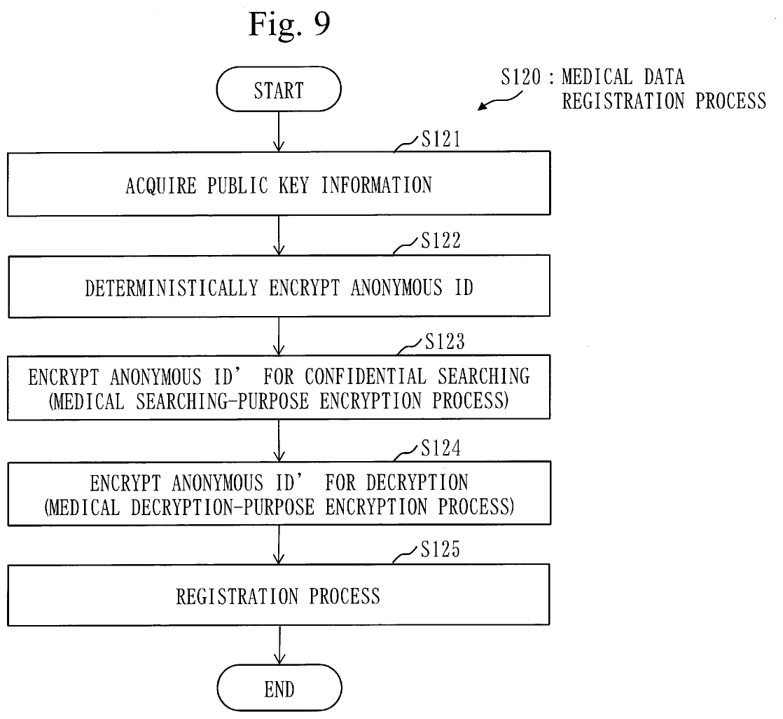

FIG. 9 is a flowchart illustrating the medical data registration process S120 according to the present embodiment.

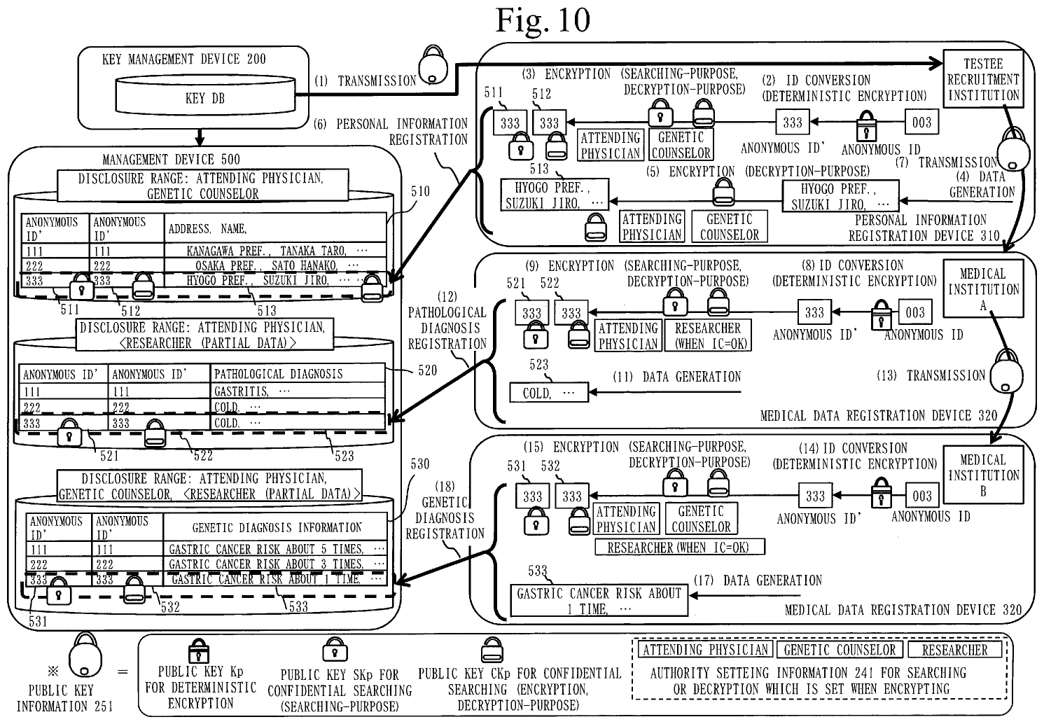

FIG. 10 is a schematic diagram illustrating the personal information registration process S110 and the medical data registration process S120.

<Personal Information Registration Process S110>

The personal information registration process S110 is executed by the personal information registration device 310.

In step S111, the public key acquisition unit 311 acquires the public key information 251 from the key management device 200. Specifically, as in (1) of FIG. 10, the key management device 200 transmits the public key information 251 to the personal information registration device 310.

In step S112, the deterministic encryption unit 312 encrypts the anonymous ID into the anonymous ID′, using the public key Kp included in the public key information 251. Step S112 corresponds to (2) of FIG. 10.

In step S113, the personal searching-purpose encryption unit 313 encrypts the anonymous ID′ as the personal searching-purpose ID 511, using the public key SKp for confidential searching and the authority setting information 241 included in the public key information 251.

In step 114, the personal decryption-purpose encryption unit 314 encrypts the anonymous ID′ and the personal information as the personal encryption ID 512 and the encrypted personal information 513, using the public key CKp for encryption and the authority setting information 241 included in the public key information 251.

Specifically, in (3) of FIG. 10, the personal searching-purpose encryption unit 313 encrypts the anonymous ID′ as the personal searching-purpose ID 511 with “attending physician” and “genetic counselor” embedded, which are the disclosure range of the personal information. Also, personal decryption-purpose encryption unit 314 encrypts the anonymous ID′ as the personal encryption ID 512 with “attending physician” and the “genetic counselor” embedded, which are the disclosure range of the personal information. Also, in (4) of FIG. 10, the personal information is generated. In (5) of FIG. 10, the personal decryption-purpose encryption unit 314 encrypts the personal information as the encrypted personal information 513 with “attending physician” and “genetic counselor” embedded, which are the disclosure range of the personal information.

In step S115, the registration unit 315 transmits the personal searching-purpose ID 511, the personal encryption ID 512 and the encrypted personal information 513 to the management device 500. Specifically, in (6) of FIG. 10, a row of the anonymous personal information 510 is registered in the management device 500. In (7) of FIG. 10, the public key information 251 is transmitted to a device of the medical institution A that is the medical data registration device 320. Note that the public key information 251 may also be transmitted from the key management device 200 to the device of the medical institution A that is the medical data registration device 320.

<Medical Data Registration Process S120>

The medical data registration process S120 is executed by the medical data registration device 320.

In step S121, the public key acquisition unit 321 acquires the public key information 251 from the personal information registration device 310.

In step S122, the deterministic encryption unit 322 encrypts the anonymous ID into the anonymous ID′, using the public key Kp. Step S122 corresponds to (8) and (14) of FIG. 10.

In step S123, the medical searching-purpose encryption unit 323 encrypts the anonymous ID′ as the medical searching-purpose ID 5011, embedding the disclosure range of the medical data, using the public key SKp for confidential searching and the authority setting information 241 included in the public key information 251.

In step S124, the medical decryption-purpose encryption unit 324 encrypts the anonymous ID′ as the medical encryption ID 5012, embedding the disclosure range of the medical data, using the public key CKp for encryption and the authority setting information 241 included in the public key information 251.

Here, the disclosure range may be decided for each of the medical searching-purpose ID 5011 and the medical encryption ID 5012 according to an informed consent (written as an IC hereinafter) indicating if the medical data may be used for a research-purpose or not.

The IC is information indicating if using the medical data by a researcher for a research-purpose is permitted (agreed) or not. That is, it is possible to decide whether or not to include the researcher who uses the medical data for the research-purpose in the disclosure range, depending on the contents of the IC. If the IC indicates a permission, encryption is performed embedding the attribution information of the researcher. On the other hand, if the IC indicates a non-permission, encryption is performed without embedding the attribution information of the researcher. At this time, the medical searching-purpose ID 5011 and the medical encryption ID 5012 are either data that can be searched for and decrypted by the researcher or data that cannot be searched for and decrypted by the researcher. By this method, each of the medical searching-purpose ID 5011 and the medical encryption ID 5012 may indicate whether or not the medical data can be used for the research-purpose. In the following description, the IC indicating an agreement or a permission may be referred to as the IC being OK. Also, the IC indicating a disagreement or a non-permission may be referred to as the IC being NG.

In the medical institution A, in (9) of FIG. 10, the medical searching-purpose encryption unit 323 encrypts the anonymous ID′ as the pathological searching-purpose ID 521 embedding “attending physician” and “researcher” having the disclosure range of the pathological diagnosis information. The medical decryption-purpose encryption unit 324 encrypts the anonymous ID′ as the pathological encryption ID 522 embedding “attending physician” and “researcher” having the disclosure range of the pathological diagnosis information. At this time, “researcher” is embedded only when the IC permits the use of pathological diagnosis information. “Researcher” is not embedded when the IC does not permit the use of the pathological diagnosis information. That is, when the IC is NG, only “attending physician” is embedded as the disclosure range. Therefore, the medical searching-purpose encryption unit 323 encrypts the anonymous ID′ as the pathological searching-purpose ID 521 embedding “attending physician” and “researcher” (only “attending physician” when the IC is NG). The medical decryption-purpose encryption unit 324 encrypts the anonymous ID′ as the pathological encryption ID 522 embedding “attending physician” and “researcher” (only “attending physician” when IC is NG).

Also in the medical institution B, in (15) of FIG. 10, the medical searching-purpose encryption unit 323 encrypts the anonymous ID′ as the genetic searching-purpose ID 531 embedding “attending physician”, “genetic counselor” and “researcher” (only “attending physician” and “genetic counselor” when the IC is NG). The medical decryption-purpose encryption unit 324 encrypts the anonymous ID′ as the genetic encryption ID 532 embedding “attending physician”, “genetic counselor” and “researcher” (only “attending physician” and “genetic counselor” when the IC is NG).

In (11) of FIG. 10, the pathological diagnosis information 523 is generated. In (17) of FIG. 10, the genetic diagnosis information 533 is generated. The medical institution B may receive the pathological diagnosis information 523 in (13) of FIG. 10 together with the public key information 251 for generating the genetic diagnosis information 533. Also, the medical institution B may receive the pathological diagnosis information 523 from the management device 500 for generating the genetic diagnosis information 533.

In step S125, the registration unit 325 transmits to the management device 500, the medical searching-purpose ID 5011, the medical encryption ID 5012 and the medical data which is not encrypted. Specifically, in (12) of FIG. 10, the registration unit 325 registers the pathological searching-purpose ID 521, the pathological encryption ID 522 and the pathological diagnosis information 523 in the management device 500 as a row of the anonymous pathological information 520. Then, in (13) of FIG. 10, the public key information 251 is transmitted to a device of the medical institution B that is the medical data registration device 320. Note that the public key information 251 may be transmitted from the key management device 200 to the device of the medical institution B that is the medical data registration device 320. Also, in (18) of FIG. 10, the registration unit 325 registers the genetic searching-purpose ID 531, the genetic encryption ID 532 and the genetic diagnosis information 533 in the management device 500 as a row of the anonymous genetic information 530.

<Searching Process S130>

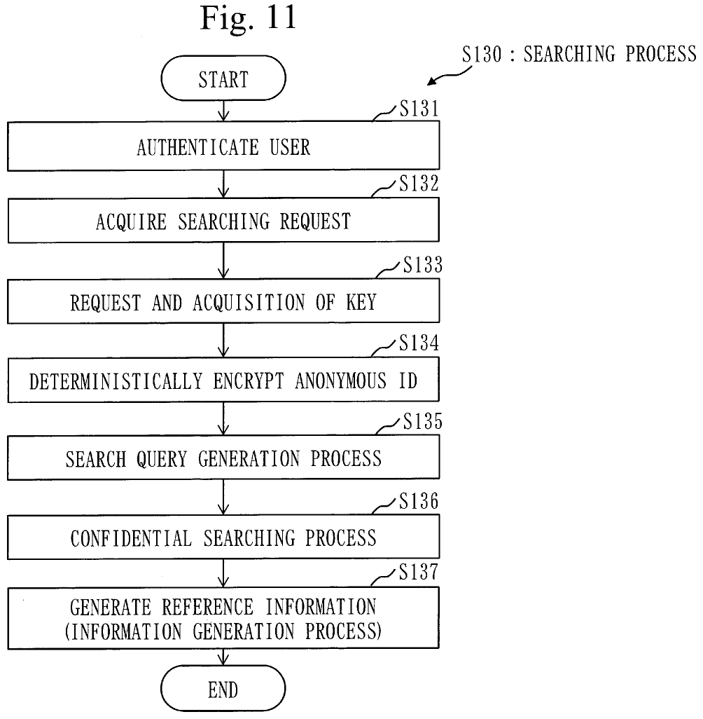

FIG. 11 is a flowchart of the searching process S130 according to the present embodiment.

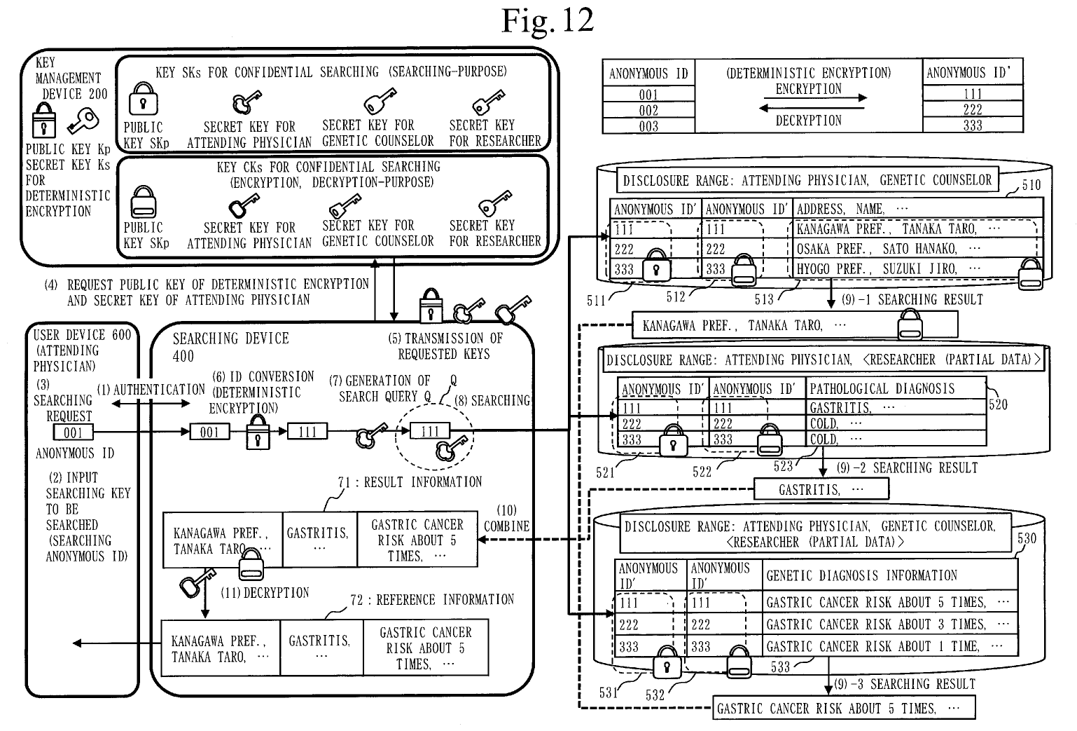

FIG. 12 is a schematic diagram illustrating a case where the attending physician searches the management device 500 as a user.

The searching process S130 is executed by the searching device 400. Here, the searching process S130 when the user is the attending physician will be described.

In step S131, the authentication unit 401 authenticates the user based on user information. Step S131 corresponds to (1) of FIG. 12.

In step S132, if authentication is successful, the attending physician that is the user inputs the search anonymous ID as a search key used for searching. The user device 600 transmits a searching request including the search anonymous ID to the searching device 400. Step S132 corresponds to (2) and (3) of FIG. 12.

In step S133, the key acquisition unit 406 requires the key management device 200 to provide the public key Kp for deterministic encryption, and the secret keys SKs and CKs corresponding to the attribution information of the user. The key acquisition unit 406 acquires the public key Kp for deterministic encryption transmitted from the key transmission unit 260 of the key management device 200, and the secret keys SKs and CKs corresponding to the attribution information of the user indicating the attribution of the user and transmitted from the key transmission unit 260 of the key management device 200. Step S133 corresponds to (4) and (5) of FIG. 12. Specifically, the key acquisition unit 406 acquires the public key Kp and the secret keys SKs, CKs corresponding to the attending physician from the key management device 200.

In step S134, the deterministic encryption unit 402 executes deterministic encryption on the search anonymous ID, using the public key Kp. Step S134 corresponds to (6) of FIG. 12.

In step S135, the search query generation unit 403 generates a search query Q with the attribution information of the user embedded, using the secret key SKs for confidential searching. In (7) of FIG. 12, the search query Q is generated in which “111” (after deterministic encryption) is embedded as the search anonymous ID, and “attending physician” is embedded as the attribution information of the user.

In step S136, the searching unit 404 executes confidential searching on the personal searching-purpose ID 511 and the medical searching-purpose ID 5011, using the search query Q. The searching unit 404 outputs the searching result acquired based on the attribution information of the user, the disclosure range of the personal information, and the disclosure range of the medical data. Specifically, as a searching result (9)-1, the searching unit 404 outputs the personal encryption ID 512 and the encrypted personal information 513 corresponding to the personal searching-purpose ID 511 in which the attribution information of the user embedded in the search query Q satisfies the disclosure range of the personal information. Also, as the searching result (9)-2 and (9)-3, the searching unit 404 outputs the medical encryption ID 5012 and the medical data corresponding to the medical searching-purpose ID 5011 in which the attribution information of the user embedded in the search query Q satisfies the disclosure range of the medical data.

In (8) and (9) of FIG. 12, the searching unit 404 searches the anonymous personal information 510, the anonymous pathological information 520 and the anonymous genetic information 530, using the search query Q of the attending physician including “111” (after deterministic encryption) as the search anonymous ID.

In the anonymous personal information 510, “attending physician” is included in the disclosure range. Therefore, the searching unit 404 extracts, as the searching result, the personal encryption ID 512 and the encrypted personal information 513 including “111” as the personal searching-purpose ID 511. Also in the anonymous pathological information 520, “attending physician” is included in the disclosure range. Therefore, the searching unit 404 extracts, as the searching result, the pathological encryption ID 522 and pathological diagnosis information 523 including “111” as the pathological searching-purpose ID 521.

Also in the anonymous genetic information 530, “attending physician” is included in the disclosure range. Therefore, the searching unit 404 extracts, as the searching result, the genetic encryption ID 532 and the genetic diagnosis information 533 including “111” as the genetic searching-purpose ID 531.

In step S137, the information generation unit 405 decrypts the personal encryption ID 512 and the medical encryption ID 5012 output as the searching result. The information generation unit 405 combines, as result information 71, the encrypted personal information 513 and the medical data output as the searching result, when decryption results of the personal encryption ID 512 and the medical encryption ID 5012 are equal. That is, the personal encryption ID 512 and the medical encryption ID 5012 are information used when combining the personal information or the medical data.

In FIG. 12, the encrypted personal information 513 of (9)-1, the pathological diagnosis information 523 of (9)-2 and the genetic diagnosis information 533 of (9)-3 are output as the searching result. In (10) of FIG. 12, the information generation unit 405 decrypts the personal encryption ID 512 of (9)-1, the pathological encryption ID 522 of (9)-2 and the genetic encryption ID 532 of (9)-3 with a secret key for the attending physician. The information generation unit 405 combines the encrypted personal information 513 of (9)-1, the pathological diagnosis information 523 of (9)-2 and the genetic diagnosis information 533 of (9)-3 as result information 71, when all of decryption results of the personal encryption ID 512 of (9)-1, the pathological encryption ID 522 of (9)-2 and the genetic encryption ID 532 of (9)-3 are “111”. The information generation unit 405 decrypts the result information 71 into reference information 72, using the secret key CKs of the attending physician for encryption. In (11) of FIG. 12, the information generation unit 405 decrypts the encrypted personal information 513 out of the result information 71 into a plaintext. The pathological diagnosis information 523 and the genetic diagnosis information 533 remain plaintext. Then, the information generation unit 405 transmits the reference information 72 to the user device 600 of the attending physician.

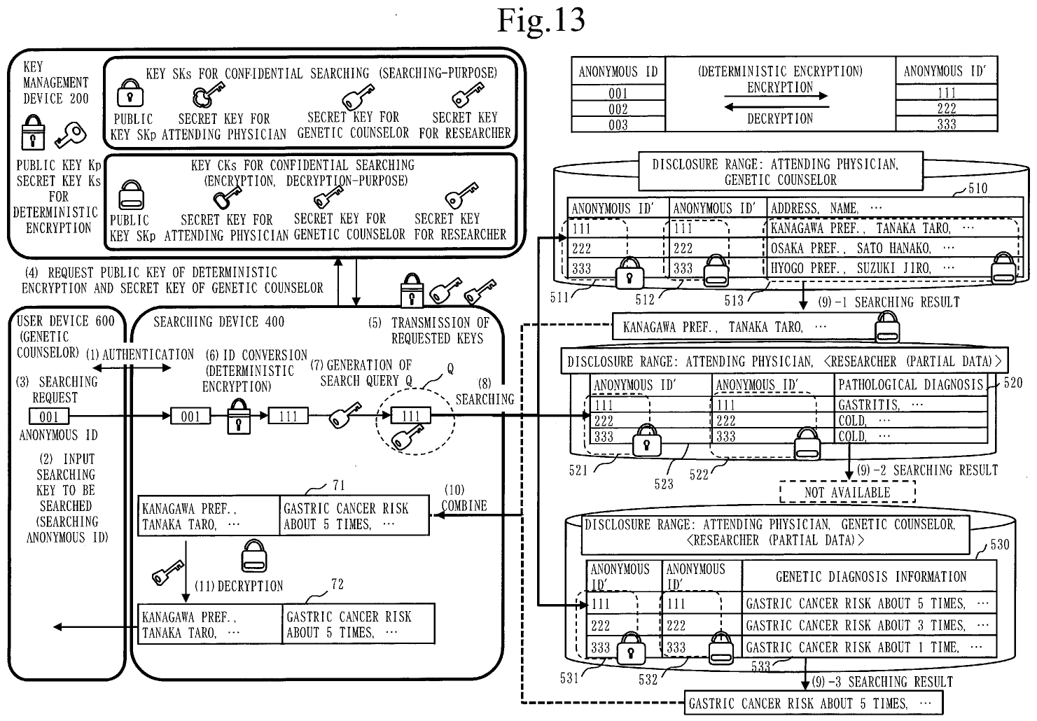

Next, a case where the genetic counselor as a user searches the management device 500 will be described, using FIG. 13.

In (1) of FIG. 13, the authentication unit 401 authenticates the genetic counselor who is the user.

In (2) and (3) of FIG. 13, the genetic counselor who is the user inputs the anonymous ID as the search key for searching. The user device 600 transmits a searching request including the search anonymous ID to the searching device 400.

In (4) and (5) of FIG. 13, specifically, the key acquisition unit 406 acquires the public key Kp and the secret keys SKs and CKs corresponding to the genetic counselor from the key management device 200.

In (6) of FIG. 13, the deterministic encryption unit 402 executes deterministic encryption on the search anonymous ID, using the public key Kp.

In (7) of FIG. 13, the search query Q is generated with “111” (after deterministic encryption) as the search anonymous ID embedded, and “genetic counselor” as the attribution information of the user embedded.

In (8) and (9) of FIG. 13, the searching unit 404 confidentially searches the anonymous personal information 510, the anonymous pathological information 520 and the anonymous genetic information 530, using the search query Q of the genetic counselor including “111” (after deterministic encryption) as the search anonymous ID.

In the anonymous personal information 510, “genetic counselor” is included in the disclosure range. Therefore, the searching unit 404 extracts, as searching result (9)-1, the personal encryption ID 512 and the encrypted personal information 513 including “111” as the personal searching-purpose ID 511. Also, in the anonymous pathological information 520, “genetic counselor” is not included in the disclosure range. Therefore, the searching unit 404 does not hit in the anonymous pathological information 520.

Also, in the anonymous genetic information 530, “genetic counselor” is included in the disclosure range. Therefore, the searching unit 404 extracts, as the searching result (9)-3, the genetic encryption ID 532 and the genetic diagnosis information 533 including “111” as the genetic searching-purpose ID 531.

In FIG. 13, the encrypted personal information 513 of (9)-1 and the genetic diagnosis information 533 of the (9)-3 are output as the searching result.

In (10) of FIG. 13, the information generation unit 405 combines the encrypted personal information 513 of (9)-1 and the genetic diagnosis information 533 of (9)-3 as the result information 71, when all of decryption results of the personal encryption ID 512 of (9)-1 and the genetic encryption ID 532 of (9)-3 are “111”.

In (11) of FIG. 13, the information generation unit 405 decrypts the result information 71 into the reference information 72, using the secret key CKs of the genetic counselor for encryption. Then, the information generation unit 405 transmits the reference information 72 to the user device 600 of the genetic counselor.

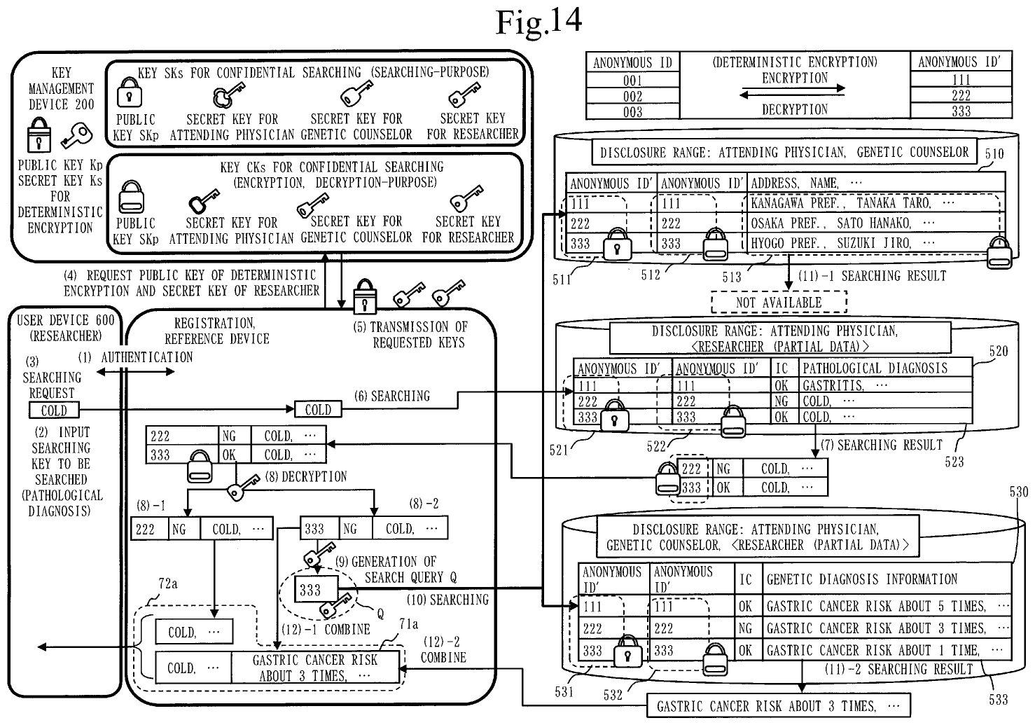

Next, a case where the researcher as a user searches the management device 500 will be described, using FIG. 14. To simplify explanation, in the anonymous pathological information 520 and the anonymous genetic information 530 in FIG. 14, it is indicated if the IC is OK or NG. That is, “researcher” is embedded as the disclosure range when the IC is OK, but “researcher” is not embedded as the disclosure range when the IC is NG.

In (1) of FIG. 14, the authentication unit 401 authenticate the researcher who is the user.

In (2) and (3) of FIG. 14, the researcher who is the user inputs the pathological diagnosis as the search key for searching. The user device 600 transmits a searching request including the pathological diagnosis to the searching device 400. Here, it is assumed that “COLD” is input as the pathological diagnosis the researcher wants to research.

In (4) and (5) of FIG. 14, specifically, the deterministic encryption unit 402 acquires the public key Kp and the secret keys SKs and CKs corresponding to the researcher from the key management device 200. Note that there is no need to acquire the public key Kp when the researcher searches using the pathological diagnosis or the genetic diagnosis as the search key instead of the anonymous ID.

In (6) of FIG. 14, the searching unit 404 searches the anonymous pathological information 520 with “COLD” as the search key. The searching unit 404 extracts rows of pathological diagnosis information 523 which include “COLD”. In the extracted rows, the pathological searching-purpose ID 521, the pathological encryption ID 522 and the pathological diagnosis information 523 are included.

In (6) of FIG. 14, the searching unit 404 executes simple searching with the pathological diagnosis as the search key instead of confidential searching. Therefore, the searching unit 404 extracts all the rows of the pathological diagnosis information 523 which include “COLD”. In (7) of FIG. 14, the searching unit 404 extracts the row in which the anonymous ID′ is “222” the IC is NG, and the row in which the anonymous ID′ is “333” and the IC is OK.

In (8) of FIG. 14, the information generation unit 405 decrypts the anonymous ID′ (pathological encryption ID 522) of the extracted rows by the secret key CKs of the researcher for encryption. At this time, as in (8)-1 of FIG. 14, “researcher” is not embedded in the pathological encryption ID 522 of the row in which the IC is NG, and hence the anonymous ID′ cannot be decrypted. In (8)-2 of FIG. 14, the IC is OK, and “researcher” is embedded in the pathological encryption ID 522, and hence the anonymous ID′ can be decrypted.

In (9) of FIG. 14, the search query Q is generated in which “333” being the decrypted anonymous ID′ is embedded, and “researcher” is embedded as the attribution information of the user.

In (10) and (11) of FIG. 14, the searching unit 404 confidentially searches the anonymous personal information 510 and the anonymous genetic information 530, using the search query Q in which “333” as the anonymous ID′ and “researcher” as the disclosure range are embedded.

In the anonymous personal information 510, “researcher” is not included in the disclosure range. Therefore, as in (11)-1 of FIG. 14, the searching unit 404 does not hit in the anonymous personal information 510.

Also, in the anonymous genetic information 530, “researcher” whose IC is OK is included in the disclosure range. Therefore, as in (11)-2 of FIG. 14, the searching unit 404 extracts, as the searching result, the genetic encryption ID 532 and the genetic diagnosis information 533 of the row in which the genetic searching-purpose ID 531 is “333”. The row in which the genetic searching-purpose ID 531 is “222” is not extracted as the IC is NG and “researcher” is not embedded in the genetic encryption ID 532.

In FIG. 14, the pathological diagnosis information 523 of (8)-1, the pathological encryption ID 522 and the pathological diagnosis information 523 of (8)-2, and the genetic encryption ID 532 and the genetic diagnosis information 533 of (11)-2 are output as the searching result.

In (12) of FIG. 14, the information generation unit 405 combines the pathological diagnosis information 523 of (8)-2 and the genetic diagnosis information 533 of (11)-2 as result information 71a, when decryption results of the pathological encryption ID 522 of (8)-2 and the genetic encryption ID 532 of (11)-2 are equal.

Since the anonymous ID of the pathological diagnosis information 523 of (8)-1 is unknown, it cannot be combined with other pieces of information.

Then, the searching device 400 transmits to the user device 600 of the researcher, the result information 71 together with the pathological diagnosis information 523 of (8)-1, as reference information 72a.

***Other Configuration***

In the present embodiment, functions of “units” of each device of the medical data searching system 100 are realized by software, but as a variation, the functions of “units” of each device of the medical data searching system 100 may be realized by hardware. Each device of the medical data searching system 100 may include a processing circuit in place of the processor 901.

The processing circuit is an exclusive electric circuit realizing the functions of “units” of each device described above.

The processing circuit is specifically a single circuit, a composite circuit, a programmed processor, a parallel-programmed processor, a logic IC, a GA (Gate Array), an ASIC (Application Specific Integrated Circuit) or an FPGA (Field-Programmable Gate Array).

The functions of “units” of each device of the medical data searching system 100 may be realized by one processing circuit, or may be realized separately by a plurality of processing circuits.

As another variation, the functions of “units” of each device of the medical data searching system 100 may be realized by a combination of software and hardware. That is, some of the functions of each device may be realized by exclusive hardware, and the rest of the functions may be realized by software.

The processor 901, a storage device 920 and the processing circuit are collectively referred to as “processing circuitry”. That is, the functions of “units” of each device of the medical data searching system 100 are realized by the processing circuitry.

“Units” may be read as “steps”, “procedures” or “processes”. Also, the functions of “units” may be realized by firmware.

***Description of Effect of Embodiment***

In the medical data searching system 100 according to the present embodiment, an anonymous ID is correlated to personal information and medical data stored in a management device, the anonymous ID being encrypted by a confidential searching technology with a disclosure range embedded. Therefore, according to the medical data searching system 100 of the present embodiment, it is possible to perform confidential searching with access control while keeping the anonymous ID encrypted. Consequently, a partial disclosure of data or a partial concatenation of data depending on a user is enabled. Also, key management and ciphertext management are not complicated, which reduces burdens of management.

In the medical data searching system 100 according to the present embodiment, the personal information and the medical data can be registered in the management device, based on authority setting information in which the disclosure range of the personal information and the medical data is set. Therefore, according to the medical data searching system 100 of the present embodiment, a change of the disclosure range of the personal information and the medical data is facilitated.

In the medical data searching system 100 according to the present embodiment, information can be encrypted according to an IC which indicates whether the medical data can be used for a research-purpose or not. Therefore, according to the medical data searching system 100 of the present embodiment, fine access control is enabled.

In the medical data searching system 100 according to the present embodiment, the anonymous ID for each of confidential-purpose and decryption-purpose is encrypted by the confidential searching technology with access control. Therefore, according to the medical data searching system 100 of the present embodiment, high security and appropriate access control is enabled.

In the present embodiment, a case is described where a medical data searching system includes a key management device, a personal information registration device, a medical data registration device, a searching device and the management device, and each device is one computer. However, for example, the key management device and the personal information registration device may be in one computer. Also, the searching device and the management device may be in one computer. Also all the devices may be realized by one computer. As long as the functions described in the embodiment above are realized, the medical data searching system may be composed by any combination of devices of the medical data searching system.

In each device of the medical data searching system, only one of those described as “units” may be adopted, or an arbitrary combination of some may be adopted. That is, any functional block may be employed in each device of the medical data searching system as long as the functions described in the embodiment above can be realized. Any combination of these functional blocks may be employed to compose to each device.

A plurality of portions of this embodiment may be implemented in combination. Alternatively, one invention of this embodiment may be implemented partially. Besides, this embodiment may be implemented as a whole or partially in any combination.

The above-described embodiment is essentially preferable exemplification, and is not intended to limit the scope of the present invention, and the scope of applications and intended use of the present invention, and various modifications are possible as necessary.

### REFERENCE SIGNS LIST

100: medical data searching system; 200: key management device; 210: deterministic key storage unit; 220: confidential searching-purpose key storage unit; 230: encryption-purpose key storage unit; 240: information storage unit; 241: authority setting information; 250: public key information transmission unit; 251: public key information; 260: key transmission unit; 310: personal information registration device; 311, 321: public key acquisition unit; 312, 322, 402: deterministic encryption unit; 313: personal searching-purpose encryption unit; 314: personal decryption-purpose encryption unit; 315, 325: registration unit; 320: medical data registration device; 323: medical searching-purpose encryption unit; 324: medical decryption-purpose encryption unit; 400: searching device; 401: authentication unit; 403: search query generation unit; 404: searching unit; 405: information generation unit; 406: key acquisition unit; 500: management device; 600: user device; 51: personal information storage unit; 501: medical data storage unit; 52: pathological information storage unit; 53: genetic information storage unit; 510: anonymous personal information; 511: personal searching-purpose ID; 512: personal encryption ID; 513: encrypted personal information; 520: anonymous pathological information; 521: pathological searching-purpose ID; 522: pathological encryption ID; 523: pathological diagnosis information; 530: anonymous genetic information; 531: genetic searching-purpose ID; 532: genetic encryption ID; 533: genetic diagnosis information; 5011: medical searching-purpose ID; 5012: medical encryption ID; 610: medical data searching method; 620: medical data searching program; 71, 71a: result information; 72, 72a: reference information; 901: processor; 902: auxiliary storage device; 903: memory; 904: communication device; 9041: receiver; 9042: transmitter; 905: input interface; 906: output interface; 907: input device; 908: output device; 911, 912: cable; S100: medical data searching process; S110: personal information registration process; S120: medical data registration process; S130: searching process; Q: search query; Kp, SKp, CKp: public key; Ks, SKs, CKs: secret key.

1-12. (canceled)

13. A data searching system comprising:
a management device including a memory:
to store a personal searching-purpose ID, a personal encryption ID and encrypted personal information, the personal searching-purpose ID being a personal searching-purpose ID used for confidential searching and obtained by encrypting an anonymous ID (IDentifier) for identifying personal information with a disclosure range of the personal information embedded, the personal encryption ID and the encrypted personal information being obtained by encrypting the anonymous ID and the personal information with the disclosure range of the personal information embedded; and
to store a received searching-purpose ID, a received encryption ID and received data, the received searching-purpose ID being a received searching-purpose ID used for confidential searching and obtained by encrypting the anonymous ID with a disclosure range of the received data received by a person corresponding to the personal information embedded, the received encryption ID being obtained by encrypting the anonymous ID with the disclosure range of the received data embedded; and
a searching device including processing circuitry:
to acquire the anonymous ID subject to searching from a user as a search anonymous ID, and generate a search query obtained by encrypting the search anonymous ID with attribution information of the user embedded; and
to execute confidential searching on the personal searching-purpose ID and the received searching-purpose ID, using the search query, and output a searching result acquired based on the attribution information of the user, the disclosure range of the personal information and the disclosure range of the received data.

14. The data searching system according to claim 13,
wherein the received data is medical data received by the person,
wherein the received searching-purpose ID is a medical searching-purpose ID used for confidential searching and obtained by encrypting the anonymous ID with a disclosure range of the medical data embedded, and
wherein the received encryption ID is a medical encryption ID obtained by encrypting the anonymous ID with the disclosure range of the medical data embedded.

15. The data searching system according to claim 14 comprising:
a key management device including
a memory to store a confidential searching-purpose public key and a confidential searching-purpose secret key with the attribution information of the user embedded,
to store an encryption-purpose public key and an encryption-purpose secret key with the attribution information of the user embedded, and
to store authority setting information including the disclosure range of the personal information and the disclosure range of the medical data; and
processing circuitry to transmit public key information including the confidential searching-purpose public key, the encryption-purpose public key and the authority setting information.

16. The data searching system according to claim 15 comprising:
a personal information registration device including processing circuitry:
to encrypt the anonymous ID as the personal searching-purpose ID with the disclosure range of the personal information embedded, using the confidential searching-purpose public key and the authority setting information included in the public key information; and
to encrypt the personal information and the anonymous ID as the encrypted personal information and the personal encryption ID with the disclosure range of the personal information embedded, using the encryption-purpose public key and the authority setting information included in the public key information.

17. The data searching system according to claim 15 comprising:
a medical information registration device including processing circuitry:
to encrypt the anonymous ID as the medical searching-purpose ID with the disclosure range of the medical data embedded, using the confidential searching-purpose public key and the authority setting information included in the public key information; and
to encrypt the anonymous ID as the medical encryption ID with the disclosure range of the medical data embedded, using the encryption-purpose public key and the authority setting information included in the public key information.

18. The data searching system according to claim 15,
wherein the processing circuitry of the searching device generates the search query with the attribution information of the user embedded, using the confidential searching-purpose secret key.

19. The data searching system according to claim 18,
wherein the processing circuitry of the searching device outputs the personal encryption ID and the encrypted personal information corresponding to the personal searching-purpose ID in which the attribution information of the user embedded in the search query satisfies the disclosure range of the personal information, as the searching result.

20. The data searching system according to claim 18,
wherein the processing circuitry of the searching device outputs the medical encryption ID and the medical data corresponding to the medical searching-purpose ID in which the attribution information of the user embedded in the search query satisfies the disclosure range of the medical data, as the searching result.

21. The data searching system according to claim 20,
wherein the memory of the management device stores the medical searching-purpose ID, the medical encryption ID and the medical data, the medical searching-purpose ID being the medical searching-purpose ID obtained by encrypting the anonymous ID, and indicating if the medical data may be used for a research-purpose or not, the medical encryption ID being the medical encryption ID obtained by encrypting the anonymous ID, and indicating if the medical data may be used for a research-purpose or not.

22. The data searching system according to claim 20 comprising:
the processing circuitry of the searching device to decrypt the personal encryption ID and the medical encryption ID output as the searching result, and combine as result information, the personal encryption ID and the medical encryption ID output as the searching result, when decrypting results of the personal encryption ID and the medical encryption ID are equal.

23. The data searching system according to claim 22,
wherein the processing circuitry of the searching device decrypts the result information to reference information, using the encryption-purpose secret key.

24. A data searching method comprising:
storing a personal searching-purpose ID, a personal encryption ID and encrypted personal information, the personal searching-purpose ID being a personal searching-purpose ID used for confidential searching and obtained by encrypting an anonymous ID (IDentifier) for identifying personal information with a disclosure range of the personal information embedded, the personal encryption ID and the encrypted personal information being obtained by encrypting the anonymous ID and the personal information with the disclosure range of the personal information embedded;
storing a received searching-purpose ID, a received encryption ID and received data, the received searching-purpose ID being a received searching-purpose ID used for confidential searching and obtained by encrypting the anonymous ID with a disclosure range of the received data received by a person corresponding to the personal information embedded, the received encryption ID being obtained by encrypting the anonymous ID with the disclosure range of the received data embedded;
acquiring the anonymous ID subject to searching from a user as a search anonymous ID, and generating a search query obtained by encrypting the search anonymous ID with attribution information of the user embedded; and
executing confidential searching on the personal searching-purpose ID and the received searching-purpose ID, using the search query, and outputting a searching result acquired based on the attribution information of the user, the disclosure range of the personal information and the disclosure range of the received data.

25. A non-transitory computer readable medium storing a data searching program for a searching device,
the searching device, that is a computer, searching in a memory of a management device storing a personal searching-purpose ID, a personal encryption ID and encrypted personal information, the personal searching-purpose ID being a personal searching-purpose ID used for confidential searching and obtained by encrypting an anonymous ID (IDentifier) for identifying personal information with a disclosure range of the personal information embedded, the personal encryption ID and the encrypted personal information being obtained by encrypting the anonymous ID and the personal information with the disclosure range of the personal information embedded, and storing a received searching-purpose ID, a received encryption ID and received data, the received searching-purpose ID being a received searching-purpose ID used for confidential searching and obtained by encrypting the anonymous ID with a disclosure range of the received data received by a person corresponding to the personal information embedded, the received encryption ID being obtained by encrypting the anonymous ID with the disclosure range of the received data embedded,
the data searching program causing the searching device to execute:
a search query generation process of acquiring the anonymous ID subject to searching from a user as a search anonymous ID, and generating a search query obtained by encrypting the search anonymous ID with attribution information of the user embedded; and
a confidential searching process of executing confidential searching on the personal searching-purpose ID and the received searching-purpose ID, using the search query, and outputting a searching result acquired based on the attribution information of the user, the disclosure range of the personal information and the disclosure range of the received data.
